(19) United States
(12) Patent Application Publication
(10) Pub. No.: US 2004/0199066 A1
(43) Pub. Date: Oct. 7, 2004

(54) Apparatus and system for providing image generator program

(76) Inventors: Naoyuki Kawachi, Tokyo (JP); Tetsuya Horiuchi, Tokyo (JP)

Correspondence Address: Patrick W. Rasche, Armstrong Teasdale LLP, Suite 2600, One Metropolitan Square, St. Louis, MO, 63102, US

(21) Appl. No.: 10/816,625

(22) Filed: Apr. 2, 2004

(30) Foreign Application Priority Data
Apr. 3, 2003 (JP) 2003-099937

(51) Int. Cl. A61B 5/05

(52) U.S. Cl. 600/407; 709/217

(57) ABSTRACT

To provide a medical program for generating desired images, a medical information provider apparatus in a service centre send to a communication processor in a hospital as a client through a network a plurality of images each corresponding to a parameter setting value as the processing result of the program by differing the parameter setting values. The communication processor selects an image desired among a plurality of images and sends to the medical information provider apparatus the selection. The medical information provider apparatus then provides the communication processor in the hospital with a program having parameter settings corresponding to the selection by the client, so as to install in an image reconstructing device to execute.

### BACKGROUND OF THE INVENTION

The present invention is related to a method for generating image generator program, apparatus and system for providing an image generator program, for providing an image generator program served for generating images by image processing based on captured data obtained from a subject by for example a CT (computed tomography scanning) apparatus or an MRI (magnetic resonance imaging) apparatus.

In medical field, there are known medical image processing apparatus such as a CT or an MRI, which executes an image generator program having setting values of parameters configured to obtain predetermined image display characteristics based on the captured data of a subject to generate medical images.

Some examples of parameters include, parameters used for the image reconstruction processing in a CT or an MRI, and coefficient of edge enhancement processing of an image.

When the manufacturer of a medical image processing apparatus have developed an update version of the medical program or a new program, the manufacturer often distributes such image generator program having predetermined parameter preconfigured to its clients for example hospitals using the medical image processing apparatus (CT and MRI).

The client then installs thus provided image generator program having predetermined parameters configured into the medical image processing apparatus to make the apparatus generate images of predetermined image display characteristics in order to diagnose based on thus obtained images.

The setting values of parameters are so far factory set by the manufacturer. The parameter setting by the user may involve a very complicate work, as well as it is often difficult to achieve the optimum parameter setting so as to have the desired image display characteristics. The improvement of such inconvenience is strongly desired.

On the other hand, the manufacturer for example, desires to provide an image generator program allowing to generate images close to the preference of clients.

### SUMMARY OF THE INVENTION

Therefore, an object of the present invention is to overcome the above problems and to provide a method for generating an image generator program, and an apparatus and system for providing an image generator program, for providing an image generator program allowing to generate desired images.

In order to achieve the above described object, a first aspect of the present invention may have a method of generating an image generator program, used for sending the image generator program, based on parameters with which different images will be reconstructed when setting different values thereto, and captured data obtained from a subject for generating an image of the subject, from an originator apparatus that is a provider of the image generator program, to a receiver apparatus that is a destination of the image generator program, comprising: a first step of sending from the originator apparatus to the receiver apparatus a plurality of the images generated by performing the image generator program with different setting values of the parameters; a second step of selecting desired images selected from the plurality of images received in the first step and sending back the result of selection to the originator apparatus from the receiver apparatus; and a third step of sending from the originator apparatus to the receiver apparatus the image generator program with setting values of the parameters configured to comply with the result of selection received in the second step.

In accordance with the first aspect of the present invention, in the first step the originator apparatus may send a plurality of images generated by executing the image generator program with different setting values of the parameters to the receiver apparatus.

In the second step the receiver apparatus may select some desired images from the plurality of images received in the first step and send back to the originator apparatus the result of selection.

In the third step the originator apparatus may send to the receiver apparatus the image generator program having the setting values of parameters configured to comply with the selection result received in the second step.

In addition, in order to achieve the object, a second aspect of the present invention include a method for generating an image generator program, used for sending the image generator program, based on parameters with which a different image will be reconstructed when setting different values thereto, and captured data obtained from a subject for generating an image of the subject, from an originator apparatus that is a provider of the image generator program, to a receiver apparatus that is a destination of the image generator program, which includes a first step of sending from the receiver apparatus to the originator apparatus, captured data obtained from the subject; a second step of sending from the originator apparatus to the receiver apparatus a plurality of the images generated by executing the image generator program with different setting values of the parameters based on the captured data received in the first step; a third step of selecting by the receiver apparatus, desired images from the plurality of images received in the second step and sending the result of selection to the originator apparatus; and a fourth step of sending from the originator apparatus to the receiver apparatus the image generator program with the setting values of parameters configured to comply with the result of selection received in the third step.

In addition, in order to achieve the object, a third aspect of the present invention includes an image generator program providing apparatus, used for sending an image generator program for generating images of a subject based on parameters that a different setting value may generate a different image and captured data obtained from the subject, to a receiver apparatus that is the destination of the image generator program, including a communication means for communicating with the receiver apparatus; and a controller means for sending to the receiver apparatus through the communication means a plurality of the images generated by executing the image generator program with different setting values of the parameters, and for sending, in accordance with the result of selection received through the communication means on the images desired by the receiver apparatus, the image generator program having the setting values of the parameters configured to comply with the selection result to the receiver apparatus through the communication means.

In addition, in order to achieve the object, a fourth aspect of the present invention includes an image generator program providing apparatus, used for sending an image generator program for generating images of a subject based on parameters that a different setting value may generate a different image and captured data obtained from the subject, to a receiver apparatus that is the destination of the image generator program, including: a communication means for communicating with the receiver apparatus; and a controller means for sending to the receiver apparatus through the communication means a plurality of images generated by executing the image generator program with captured data received from the receiver apparatus through the communication means and with various setting values of the parameters configured differently, and for sending, in accordance with the result of selection received through the communication means on the images desired by the receiver apparatus, the image generator program having the setting values of the parameters configured to comply with the selection result to the receiver apparatus through the communication means.

In addition, in order to achieve the object, a fifth aspect of the present invention includes an image generator program providing system, for sending an image generator program for generating images of a subject based on parameters that a different setting value may generate a different image and captured data obtained from the subject, from an originator apparatus that is a provider of the image generator program to a receiver apparatus that is the destination of the image generator program, in which: the originator apparatus includes: a first controller means for sending to the receiver apparatus a plurality of the images generated by executing the image generator program with different setting values of the parameters, and for sending to the receiver apparatus the image generator program having the setting values of the parameters configured to comply with the selection result received from the receiver apparatus; the receiver apparatus having: a second controller means for selecting the images desired from the plurality of images received from the originator apparatus to send the result of selection back to the originator apparatus.

In addition, in order to achieve the object, a sixth aspect of the present invention includes an image generator program providing system, for sending an image generator program for generating images of a subject based on parameters that a different setting value may generate a different image and captured data obtained from the subject, from an originator apparatus that is a provider of the image generator program to a receiver apparatus that is the destination of the image generator program, comprising: the originator apparatus having: a first controller means for sending to the receiver apparatus a plurality of the images generated by executing the image generator program with different setting values of the parameters based on the captured data received from the receiver apparatus, and for sending to the receiver apparatus the image generator program having the setting values of the parameters configured to comply with the selection result received from the receiver apparatus; the receiver apparatus having: a second controller means for sending captured data obtained from the subject to the originator apparatus and for selecting the images desired from the plurality of images received from the originator apparatus to send the result of selection back to the originator apparatus.

In accordance with the present invention, a method for generating an image generator program, an apparatus and system for providing the image generator program can be provided, which provides a medical program that may generate desired images.

Further objects and advantages of the present invention will be apparent from the following description of the preferred embodiments of the invention as illustrated in the accompanying drawings.

### BRIEF DESCRIPTION OF THE DRAWINGS

FIG. 1 is an overview of a first preferred embodiment of a medical processing system including a medical information providing device in accordance with the present invention;

FIG. 2 is a functional block diagram illustrating an exemplary medical program in accordance with the present invention;

FIG. 3 is a flowchart illustrating an operation of a medical processing system of FIG. 1;

FIG. 4 is a schematic diagram illustrating images V having selectable image display characteristics corresponding to the parameter values; and

FIG. 5 is a flowchart illustrating the operation of a medical processing system 100a in accordance with the second preferred embodiment.

### DETAILED DESCRIPTION OF THE INVENTION

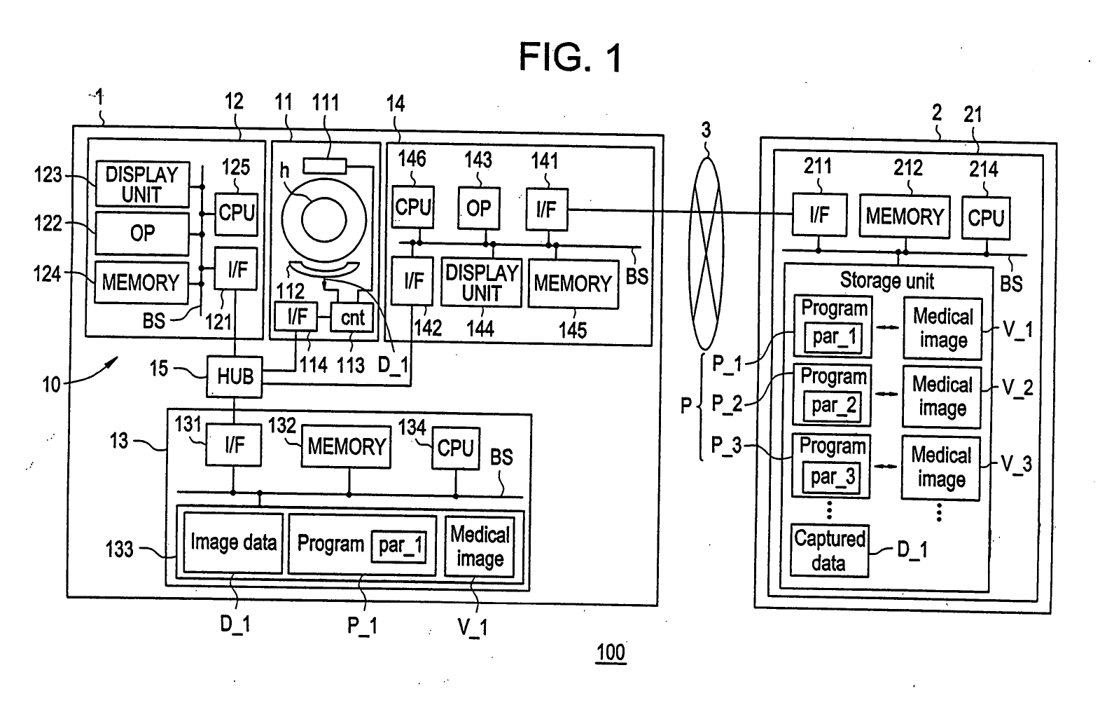

FIG. 1 is an overview of a first preferred embodiment of medical processing system including a medical information provider apparatus, which provides an image generator program in accordance with the present invention.

The medical processing system 100 may have, as shown in FIG. 1, a hospital 1 as a consumer (or a client) and a service centre 2 as a provider having a medical information providing apparatus for providing medical programs.

The medical processing system 100 may be the image generator program provider system in accordance with the present invention.

The hospital 1, as shown in FIG. 1 for example, may have a CT 10 comprised of a scanning gantry 11, operation console 12, and image reconstructing device 13, and a communication processor 14.

The scanning gantry 11, operation console 12, image reconstructing device 13, and communication processor 14 are all connected to a hub 15.

The CT 10 and the communication processor 14 may be the receiver apparatus in accordance with the present invention.

The scanning gantry 11, as shown in FIG. 1 for example, has an X-ray tube 111, detector array 112, controller 113, and interface (I/F) 114.

The X-ray tube 111 is controlled by the controller 113 to emit the X-ray of predetermined intensity to a subject h through a collimator not shown in the figure.

The detector array 112 is composed of a plurality of X-ray detector elements, which may be radiation detecting elements collimated in an array (matrix) in the directions of width and depth of X-ray beam in a sector form by the collimator not shown in the figure. The detector array 112 will output signals conforming to the detected X-ray intensity to the controller 113.

The controller 113 controls for example the X-ray tube 111, detector array 112, a revolving driver unit not shown in the figure for rotational drive of the X-ray tube 111 and detector array 112, and a table driving unit for driving the scanning table not shown in the figure and carrying the subject h thereon, based on the control signals output from the operation console 12 through the hub 15 and the interface 114.

The controller 113 also outputs the raw data, which is the image data detected by the detector array 112, to the operation console 12 through the interface 114 and the hub 15.

The interface 114 under the control of the controller 113 may communicate with the operation console 12 through the hub 15.

The operation console 12 may perform, for example, processing related to the CT scanning of the scanning gantry 11 and direct the image reconstructing device 13 to perform the reconstruction processing.

The operation console 12 may have, as shown in FIG. 1, an interface 121, an operating unit (OP) 122, a display 123, a memory 124, and a CPU (central processing unit) 125. These components are connected throrugh a bus BS.

The interface 121 under the control of the CPU 125 communicates with the scanning gantry 11, image reconstructing device 13 and communication processor 14 through the hub 15.

The operating unit 122 may output to the CPU 125 signals according to the operator's operation or to the operation on the scanning gantry 11.

The display unit 123 under the control of the CPU 125 may display an image for manipulating the scanning gantry 11, or a medical image such as the medical image generated in the image reconstructing device 13.

The memory 124 stores the captured data (raw data) and the medical image generated by the image reconstructing device 13. The memory 124 is comprised of for example RAM (random access memory), ROM (read only memory), and an HDD (hard disk drive).

The CPU 125 performs processing related to the CT scanning of the scanning gantry 11 and directs the image reconstructing device 13 to carry out a reconstruction processing in accordance with the command input from the operating unit 122.

The image reconstructing device 13 performs an image reconstruction processing based on the captured data.

The image reconstructing device 13 has, as shown in FIG. 1, an interface (I/F) 131, a memory 132, a storage unit 133, and a CPU 134. These components are all connected through a bus BS.

The interface 131 performs data communication to and from the operation console 12 and the communication processor 14 through the hub 15 under the control of the CPU 134.

The memory 132 is composed of RAM and ROM and used for the workspace of the CPU 134.

The storage unit 133 includes captured data D_1, program P_1, and medical image V_1, etc.

The captured data D_1 may be the raw data captured by the scanning gantry 11.

The program P_1 is executed by the CPU 134 to carry out the image reconstruction processing based on the captured data D_1 obtained from the subject h to generate the medical image V_1 (also referred to as simply “image”).

The program P_1 may have some parameters configured, which may generate different images when setting different values, such as the processing parameter Par_1 related to the image display characteristics. The program P_1 performs an image reconstruction processing so as to generate a medical image having image display characteristics according to the values of processing parameter Par_1, in order to generate a medical image (image of the subject h). The medical image V_1 may be the medical image generated as a result of execution of the program P_1.

The CPU 134 may execute the program P_1, perform the image reconstruction processing based on the captured data D_1, generate the medical image V_1, and for example output the processing result to the operation console 12 through the hub 15.

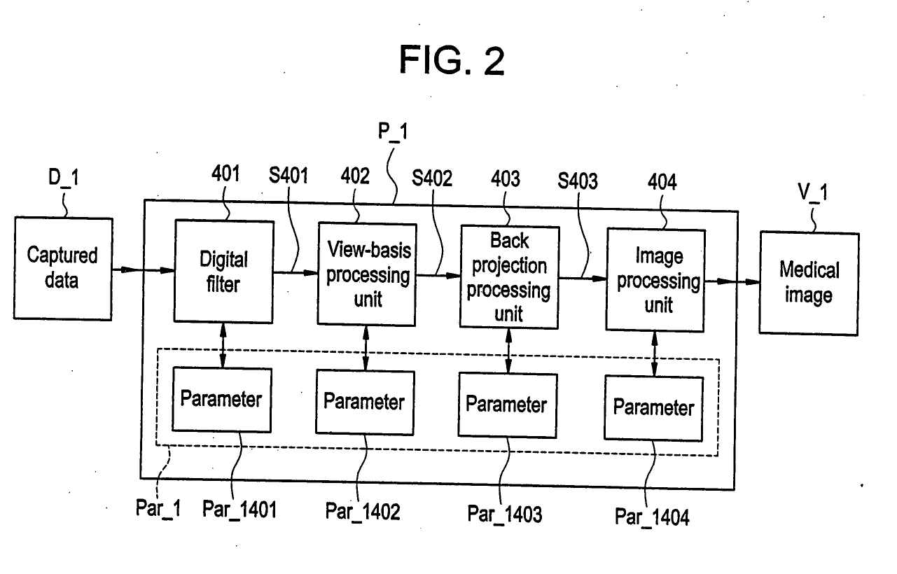

Now referring to FIG. 2, there is shown a schematic block diagram of the functions of an exemplary medical program in accordance with the present invention.

The medical program P_1 includes, as a functional block, a reconstruction processing unit 400.

The reconstruction processing unit 400 has, as shown in FIG. 2 in greater details, a digital filter 401, a view-basis processing unit 402, a back projection processing unit 403, and an image processing unit 404. The components in the reconstruction processing unit 400 performs respective processing conforming to the setting values of the processing parameter Par_1 of the predetermined image display characteristics, respectively. For example, the processing parameter Par_1 may contain parameters Par_1401 to Par_1404.

The digital filter 401 performs a high pass filtering with a cutoff frequency in accordance with the parameter Par_1401 based on the captured data (raw data) to output the processing result as signal S401 to the view-basis processing unit 402.

The view-basis processing unit 402 performs a thinning out with the thinning out coefficient K of views in accordance with the setting value of the parameter Par_1402 for example, for every views in the captured data D_1 based on the signal S401, and processing based on the matrix number in accordance with the setting value of the parameter Par_1402, to output the processing result as signal S402 to the back projection processing unit 403.

The back projection processing unit 403 performs the back projection processing in accordance with the setting value of the parameter Par_1403 based on the signal S402 to output the result as the signal S403 to the image processing unit 404.

The image processing unit 404 performs the image generating processing in accordance with the setting value of the parameter Par_1404 for example based on the back projected signal S403 to output an image (may also referred to as “image data”) V_1. The parameter Par_1404 contains an edge enhancement coefficient of the display image, a filter frequency of the bandpass filtering, and a coefficient relating to the noise reduction, etc.

The communication processor 14 performs a communication with a medical information provider apparatus 21 through a communication network 3 so as to receive a program P.

The communication processor 14 may include, for example as shown in FIG. 1, an interface 141 and 142, an operating unit 143, a display unit 144, a memory 145, and a CPU 146. These components are all connected through a bus.

The interface 141 performs communication with the medical information provider apparatus 21 through the network 3 under the control of the CPU 146.

The interface 142 performs communication with the CT 10 and the image reconstructing device 13 through the hub 15 under the control of the CPU 146.

The operating unit 143 outputs operation signals in response to the operation by a client such as an operator of the CT or a doctor, to the CPU 146.

The display unit 144 displays a medical image sent from a medical apparatus. The memory 145 is served as the workspace of the CPU 146.

The CPU 146 performs processing in accordance with the operation signals from the operating unit 143, for example selects a desired medical image among medical images, and outputs the selection result to the medical information provider apparatus 21 through the interface 141 and the network 3.

The medical information provider apparatus 21 is installed in the service centre 2 as shown in FIG. 1. The medical information provider apparatus 21 may be the originator apparatus and medical program provider apparatus in accordance with the present invention.

The medical information provider apparatus 21 may have, as shown in FIG. 1, an interface (I/F) 211, a memory 212, a storage unit 213, and a CPU 214.

The interface 211 performs communication with the communication processor 14 in the hospital (client) 1 through the network 3.

The memory 212 may be served as the workspace for the CPU 214. For example the memory 212 is comprised of RAM and ROM.

The storage unit 213 stores a medical program P, image V, captured data D_1 and so on.

The medical program P is the image generator program in accordance with the present invention.

The CPU 214 generates a medical program P for performing image reconstruction processing in a medical imaging apparatus such as the CT 10. The CPU 214 also performs the medical program P to generate the medical image (also referred to as simply “image”) V.

The medical program P includes similar function of image reconstruction processing to the medical program functional blocks shown in FIG. 2. The medical program P sets the parameter Par for the image display characteristics, and includes a procedure of image reconstruction in correspondence with the setting value of the parameter Par based on the captured data D_1.

For example, when the CPU 214 executes program P_1 having values of processing parameter Par_1 set, the program will generate the medical image V_1 based on the captured data obtained from the subject. In a similar manner, when the program P_2 is executed having values of processing parameter Par_2 set, the program will generate the medical image V_2 and when the program P_3 is executed having values of processing parameter Par_3 set, the program will generate the medical image V_3.

The CPU 214, as a procedure of determining parameter values, selects some values of most requested parameters so as to satisfy the request from the destination, among parameters to be set in a plurality of functional blocks shown in FIG. 2.

Also the CPU 214, as a procedure of selection determining parameter values, determines values of a plurality of parameters by combining parameters to be set in predetermined functional blocks among those functional blocks described above.

The CPU 214 may generate a plurality of medical images P each corresponding to a parameter value by the program P having a plurality of different values of the parameters Par set in the communication processor 14 through the interface 211 and send them to the communication processor 14 as samples.

Then based on the result of selection of the sample medical images P sent from the communication processor 14, the CPU 214 will send the program P having values of parameters Par set corresponding to the selection to provide the program to the communication processor 14.

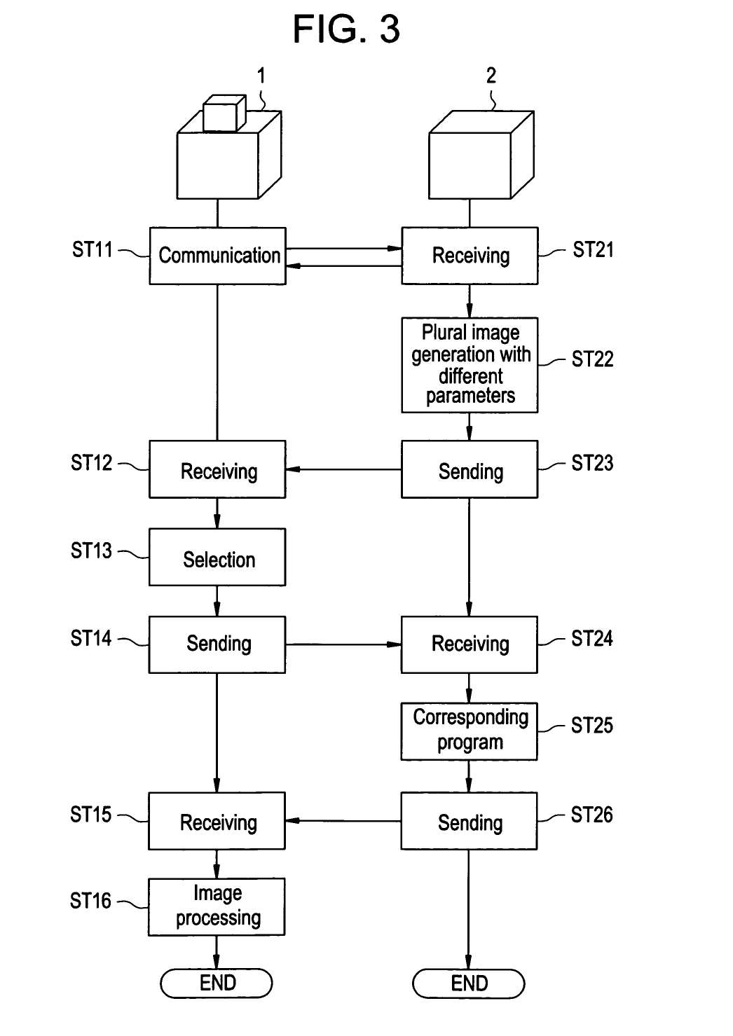

Now referring to FIG. 3, there is shown a flowchart depicting the operation of a medical processing system of FIG. 1. Now the operation of the medical processing system 100 will be described in greater details with reference to FIG. 3. For example, in the following description it is assumed that in the service centre 2, at the time of provision of a newly developed medical program to their client, they ask the client to select a setting value of parameters relating to the image display characteristics.

The communication processor 14 of the hospital 1 as a client may access the medical information provider apparatus 21 of the service centre 2 through the network 3 to obtain information with respect to a newly developed medical program P.

The medical information provider apparatus 21 provides information about the newly developed medical program P to the communication processor 14 of the previously registered client, through the network 3 (ST11, ST21).

The medical information provider apparatus 21 generates sample images V_1 to V_3 of the newly developed program P_1 to P_3 with parameters each having different values to generate different images, more specifically with parameters each having different values of the parameters Par_1 to Par_3 set to generate different image display characteristics (ST22), and sends to the communication processor 14 of the hospital 1 through the network 3 (ST23).

The communication processor 14 receives a plurality of medical images V_1 to V_3 to display on the display unit 144 (ST12), then selects a desired medical image V_1 among a plurality of medical images (ST13), and sends data indicative of the selection to the medical information provider apparatus 21 through the network 3 (ST14).

The medical information provider apparatus 21 may receive data indicative of the selection (ST24), then set to the program P the value of the parameter Par corresponding to the selection, for example the value of the processing parameter Par_1 (ST25), and send the medical program P_1 to the communication processor 14 in the hospital 1 through the network 3 (ST26).

The communication processor 14 may receive the medical program P_1 through the network 3 (ST15), then install the program into the image reconstructing device 13 for example to store in the storage unit 133.

In the image reconstructing device 13 the CPU 134 may execute the program P_1 having setting values of the parameter Par_1 set to generate images V_1 of desired image display characteristics based on the captured data (raw data) on the subject h output from the scanning gantry 11.

As have been described above, the medical information provider apparatus 21 in the service centre 2 may send a plurality of medical images V, which are the result of processing by the medical program P, each corresponding to one of setting values of the parameters Par, and each having a different setting value of the parameters, to the communication processor 14 in the hospital 1, the client, through the network 3. The communication processor 14 in turn will select desired images V among a plurality of images V and will send back the selection information to the medical information provider apparatus 21, which will provide the communication processor 14 in the hospital 1 with a program P complying with the parameter values Par corresponding to the selection result, and the program P may be installed into the image reconstructing device 13 and then executed. In such a manner a medical program to generate desired images can be provided.

The hospital 1, as a client, may select images V of desired image display characteristics among a plurality of images V sent from the service centre 2, for example, images with image display characteristics suitable to highly sensitive detection of some legions, and send the selection result to the service centre 2 to have the service of the program P provided for generating images of those image display characteristics.

The service centre 2 on the other hand may provide the clients a program P for generating images with the image display characteristics complying with the preference of the clients.

It should be noted here that although in this preferred embodiment, the service centre 2 have been described so as to provide the client 1 with a plurality of images V generated by the program P having a plurality of different parameter values set, the present invention is not intended to be limited thereto.

For example, the medical information provider apparatus 21 may also generate images V having selectable image display characteristics each corresponding to a parameter value, based on the processing result by the program P having parameter values varied, and send to the communication processor 14 of the client 1.

More specifically, images may be sent by inputting a value of a desired parameter Par, such that only the images V corresponding to the value of that parameter Par will be displayed.

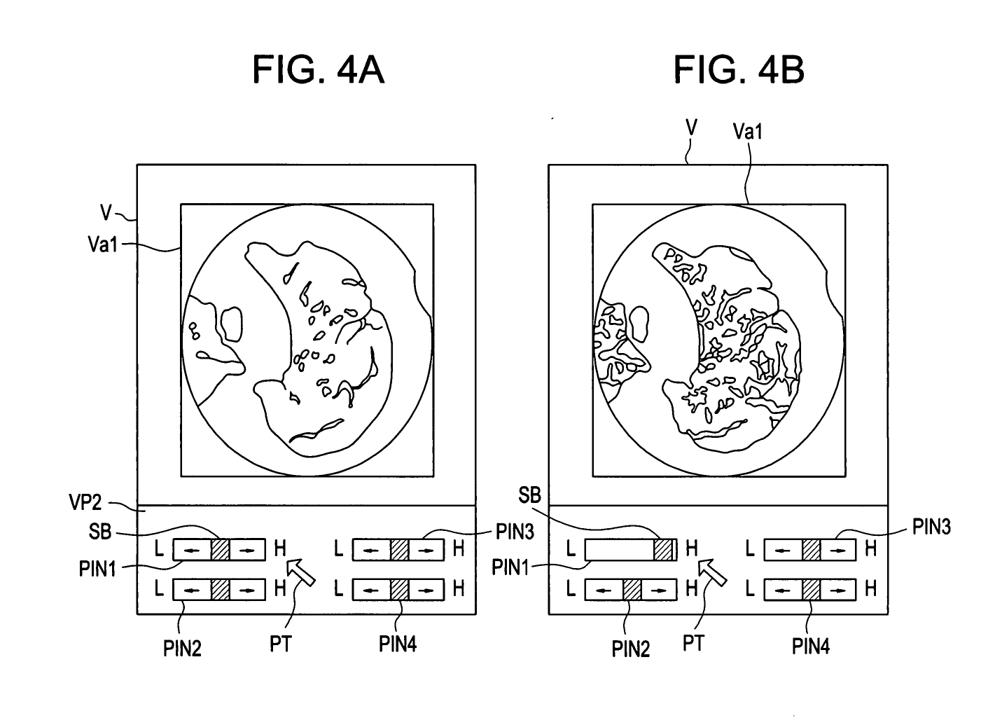

Now referring to FIGS. 4(a) and (b), there are shown schematic diagrams illustrating images V having selectable image display characteristics corresponding to the parameter values.

The communication processor 14 may receive the images V and display on the display unit only those images that correspond to the input value of the parameter Par. Then an image V may be selected having desired image display characteristics while adjusting the parameter setting values so as to send the selection result to the service centre 2.

More specifically, the image V having selectable parameter setting values contains an image display area Va1 and a parameter selection area Vp2, as shown in FIG. 4(a).

For example, in the image display area Va1 an image will be displayed with image display characteristics corresponding to the parameter setting value selected in the parameter selection area Vp2.

The parameter selection area Vp2 includes a plurality of parameter input sections PIN, for example parameter input PT1 to PT4, and a pointer PT displaceable according to the manipulation of a mouse by an operator on the operating unit 143.

In the parameter input sections PIN a desirable parameter value may be selectable according to the amount of movement to left or right direction of the sliding button SB as shown in FIG. 4.

In this preferred embodiment, the parameter input section PT1 can select the parameter setting value with respect to the edge enhancement, by sliding the sliding button SB to right to display in the image display area Va1 an image with more edge enhancement applied, as shown in FIG. 4(b).

In this manner, the client 1 may select parameter values of desired image display characteristics in an effortless manner.

A medical processing system 100a in accordance with a second preferred embodiment of the present invention may include, similar to the first preferred embodiment, although not shown in the figure, a hospital 1 as a client, and a service centre 2 having a medical information provider apparatus 21 for providing a medical program. In the hospital 1 a CT 10 and a communication processor 14 may be installed as similar to the first preferred embodiment.

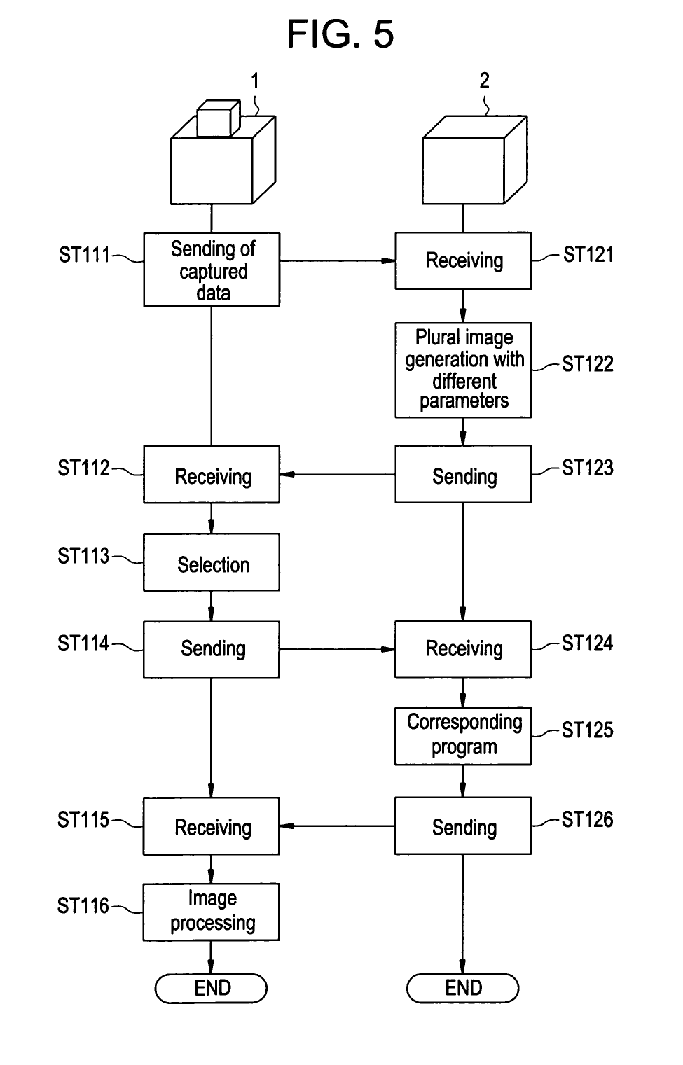

Now referring to FIG. 5, there is shown a flowchart illustrating the operation of the medical processing system 100a in accordance with the second preferred embodiment. The operation of the medical processing system 100a will be described in greater details with reference to FIG. 5. In the following only the difference will be described in greater details and the description of the components and operation similar to the preceding first preferred embodiment will be omitted.

Now an example will be described in which the hospital 1 as a client may request a medical program P for generating an image V preferable to detect desired lesions from the captured data (raw data), while the service centre 2 provides a medical program P complying with the request.

The communication processor 14 in the hospital as a client 1 outputs the captured data (raw data) obtained by the CT 10 to the medical information provider apparatus 21 in the service centre 2 through the network 3 (ST11).

The medical information provider apparatus 21 may generate medical images V_1 to V_3 that are respectively generated by new programs P_1 to P_3, each having setting values of a plurality of parameters, for example parameters Par_1 to Par_3 set with different image display characteristics based on the captured data (ST122) and send those images to the communication processor 14 in the hospital 1 through the network 3 (ST123).

The communication processor 14 receives a plurality of images V_1 to V_3, and displays on the display unit 144 (ST112). For example, a desired image V_1 is selected from a plurality of images (ST113), data indicative of the selection result will be sent to the medical information provider apparatus 21 through the network 3 (ST114).

The medical information provider apparatus 21 may receive data indicative of the selection result (ST124), set to the medical program P the setting values of parameters Par corresponding to the selection, such as setting values of parameter Par_1 (ST125), then send the program P_1 to the communication processor 14 in the hospital 1 through the network 3 (ST26).

The communication processor 14 may receive the program P_1 through the network 3 (ST15), and install for example into the image reconstructing device 13 to store in the storage unit 133.

In the image reconstructing device 13, the CPU 134 executes the program P_1 having set a setting value of the parameter Par_1 to generate an image V_1 of desired image display characteristics based on the captured data (raw data) of the subject h, output from the scanning gantry 11.

In this preferred embodiment, the hospital 1 as a client may request a medical program P for generating an image V suitable to detect desirable lesions from the captured data (raw data), and the service centre 2 in turn may provide the program P complying with the request. In this manner a medical program P much conformed to the needs of client can be provided.

Also it should be noted here that although in this preferred embodiment a program P having desired parameter setting values set is provided, the present invention is not intended to be limited thereto. For example, at the time when the medical information provider apparatus 21 provides the client with a program P having the parameter setting values with respect to desired image display characteristics, the program can be provided as a sample program with a predetermined trial period. During this period the client tries the program P and the client will send a formal request of purchase to the service centre 2 only if the client is satisfied with the new program. The service centre 2 in response to the request will send either a formal program P or a password to release the trial limit.

In this manner any possible needs among client can be elicited so that a program will be provided to the most of clients for generating images with specific image display characteristics desired by the client.

The medical information provider apparatus 21 may store the captured data sent from the client into a database not shown in the figure to perform thereon an aggregation or a statistics in order to reflect to the provision of developing program.

The medical information provider apparatus 21 may input the setting values of desirable parameter Par based on the captured data sent from the communication processor 14 to send images such that only the image V corresponding to this setting values of parameter Par will be displayed. The communication processor 14 may receive this image V and by inputting setting values of parameter Par to display only the image corresponding thereto on the display unit, then an image V having desired image display characteristics can be selected by changing the parameter setting values, and it may send the selection result to the service centre 2. In this manner the client 1 can select parameter setting values of desired image display characteristics in an easier manner.

It is to be noted that the present invention is not intended to be limited to the details and embodiments herein given, but may be modified as are suited to a particular use contemplated.

Although in this preferred embodiment the system for providing the medical program P may have as its components a hospital 1 having a medical imaging apparatus for executing the program, and a service centre 2 having the medical information provider apparatus 21 for providing the program, the present invention is not intended to be limited thereto. The service centre 2 may provide the service to the communication processor of the medical imaging apparatus operated by the client.

In this preferred embodiment the program for the image reconstruction device of a CT is provided, however the present invention is not intended to be limited thereto. For instance, the program may be for an MRI or any other type of imaging apparatuses.

The medical information provider apparatus 21 in the service centre 2 may also perform the statistic on the geographic information about the area and country that the clients reside, parameter setting values of image display characteristics that a plurality of clients has selected in order to provide the client with a program P having preset most often requested parameter values.

Although in this preferred embodiment the image reconstructing device 13 and the operation console 12 are separated, the present invention is not limited thereto. For example, the processing by the image reconstructing device 13 can be achieved also by some components in the operation console 12.

Many widely different embodiments of the invention may be configured without departing from the spirit and the scope of the present invention. It should be understood that the present invention is not limited to the specific embodiments described in the specification, except as defined in the appended claim.

1. An image generator program providing apparatus for sending an image generator program to a receiver apparatus, said image generator program configured to generate images of a subject, each said image based on parameters having a different setting value and captured data obtained from said subject, said image generator program providing apparatus comprising:
a communication device for communicating with said receiver apparatus; and
a controller device for sending to said receiver apparatus through said communication device a plurality of said images generated by executing said image generator program with different setting values of said parameters, and for sending, in accordance with the result of selection received through said communication device on the images desired by said receiver apparatus, said image generator program having the setting values of said parameters configured to comply with the selection result to the receiver apparatus through said communication device.

2. An image generator program providing apparatus according to claim 1, wherein:
said controller device generates images having image display characteristics selectable in compliance with each of setting values of said parameters based on the result of processing by said image generator program configured to have different setting values of said parameters, sends thus generated images to said receiver apparatus through said communication device, and based on the result of selection by said receiver apparatus on the images desired thereby, sends an image generator program having setting values of said parameters configured to comply with said selection result through said communication device to said receiver apparatus.

3. An image generator program providing apparatus for sending an image generator program to a receiver apparatus, said image generator program configured to generate images of a subject, each said image based on parameters having a different setting value and captured data obtained from said subject, said image generator program providing apparatus comprising:
a communication device for communicating with said receiver apparatus; and
a controller device for sending to said receiver apparatus through said communication device a plurality of images generated by executing said image generator program with captured data received from said receiver apparatus through said communication device and with various setting values of said parameters configured differently, and for sending, in accordance with the result of selection received through said communication device on the images desired by said receiver apparatus, said image generator program having the setting values of said parameters configured to comply with the selection result to the receiver apparatus through said communication device.

4. An image generator program providing apparatus, according to claim 3, wherein:
said controller device generates images having image display characteristics selectable in compliance with each of setting values of said parameters based on the captured data received from said receiver apparatus through said communication device and on the result of processing by said image generator program configured to have different setting values of said parameters, sends thus generated images to said receiver apparatus through said communication device, and based on the result of selection by said receiver apparatus on the images desired thereby, sends an image generator program having setting values of said parameters configured to comply with said selection result through said communication device to said receiver apparatus.

5. An image generator program providing apparatus according to claim 1, wherein:
said image generator program is executed on a medical imaging apparatus including an X-ray CT (computed tomography scanning) and an MRI (magnetic resonance imaging) apparatus, which at least scans said subject to generate said captured data; and said medical imaging apparatus performs a reconstruction according to said captured data to generate medical images in compliance with parameters of image display characteristics.

6. An image generator program providing apparatus according to claim 1, wherein:
said parameters includes parameter for high-pass filtering, coefficient for thinning out views, parameter for back-projection, coefficient for edge enhancement of images, filter frequency of band-pass filtering, coefficient for noise reduction, or parameter relating to the image reconstruction; and
said controller device executes said image generator program with different setting values of said parameters to generates a plurality of images, and send them to said receiver apparatus.

7. An image generator program providing system, for sending an image generator program from an originator apparatus that is a provider of the image generator program to a receiver apparatus that is the destination of the image generator program, said image generator program configured to generate images of a subject, each said image based on parameters having a different setting value and captured data obtained from said subject, said originator apparatus comprising:
a first controller device for sending to said receiver apparatus a plurality of said images generated by executing said image generator program with different setting values of said parameters, and for sending to the receiver apparatus said image generator program having the setting values of said parameters configured to comply with the selection result received from said receiver apparatus;
said receiver apparatus comprising:
a second controller device for selecting said images desired from said plurality of images received from said originator apparatus to send the result of selection back to said originator apparatus.

8. An image generator program providing system according to claim 7, wherein:
said first controller device generates images having image display characteristics selectable in compliance with each of setting values of said parameters based on the result of processing by said image generator program configured to have different setting values of said parameters, sends thus generated images to said receiver apparatus, and based on the result of selection by said receiver apparatus on the images desired thereby, sends an image generator program having setting values of said parameters configured to comply with said selection result to said receiver apparatus; and
said second controller device selects desired images based on said images received from said originator apparatus to send the result of selection back to said originator apparatus.

9. An image generator program providing system according to of claim 7, wherein:
said image generator program is executed on a medical imaging apparatus including an X-ray CT (computed tomography scanning) and an MRI (magnetic resonance imaging) apparatus, which at least scans said subject to generate said captured data; and
said medical imaging apparatus executes said image generator program based on said captured data and according to the setting values of said parameters to reconstruct images, and generate images of said subject in compliance with the setting values of said parameters.

10. An image generator program providing system according to claim 7, wherein:
said parameters includes parameter for high-pass filtering, coefficient for thinning out views, parameter for back-projection, coefficient for edge enhancement of images, filter frequency of band-pass filtering, coefficient for noise reduction, or parameter relating to the image reconstruction; and
said originator apparatus executes said image generator program with different setting values of said parameters to generates a plurality of images, and send them to said receiver apparatus.

11. An image generator program providing apparatus according to claim 3, wherein:
said image generator program is executed on a medical imaging apparatus including an X-ray CT (computed tomography scanning) and an MRI (magnetic resonance imaging) apparatus, which at least scans said subject to generate said captured data; and said medical imaging apparatus performs a reconstruction according to said captured data to generate medical images in compliance with parameters of image display characteristics.

12. An image generator program providing apparatus according to claim 3, wherein:
said parameters includes parameter for high-pass filtering, coefficient for thinning out views, parameter for back-projection, coefficient for edge enhancement of images, filter frequency of band-pass filtering, coefficient for noise reduction, or parameter relating to the image reconstruction; and
said controller device executes said image generator program with different setting values of said parameters to generates a plurality of images, and send them to said receiver apparatus.
